(19) United States
(12) Patent Application Publication
(10) Pub. No.: US 2020/0187854 A1
(43) Pub. Date: Jun. 18, 2020

(54) METHOD FOR IDENTIFYING AND LOCATING NERVES

(71) Applicant: METAL INDUSTRIES RESEARCH & DEVELOPMENT CENTRE, Kaohsiung City 811 (TW)

(72) Inventors: Bing-Feng Huang, Kaohsiung City (TW); Ming-Hui Chen, Kaohsiung City (TW); Ting-Li Shen, Taichung City (TW); Yu-Han Shen, Taichung City (TW); Hsiang-Hsiang Choo, Taipei City (TW); Tzyy-Ker Sue, Kaohsiung City (TW)

(21) Appl. No.: 16/220,107

(22) Filed: Dec. 14, 2018

(51) Int. Cl. A61B 5/00; A61B 8/00

(52) U.S. Cl. CPC A61B 5/4893; A61B 17/58; A61B 8/4227

(57) ABSTRACT

A method for identifying and locating at least one nerve in a target region is disclosed. In the method, an image detector is used to scan the target region according to a predicted nerve trend from a nerve identification program and capture 2D cross-sectional images of subregions of the target region, and the nerve identification program is utilized to identify' whether the 2D cross-sectional images are captured of the target nerve. When the nerve identification program identifies the 2D cross-sectional images are the images of the target nerve, a 3D image showing a 3D nerve structure is constructed from the 2D cross-sectional images based on 3D image reconstruction technique.

### FIELD OF THE INVENTION

This invention generally relates to a method for identifying and locating nerves, and more particularly to a method for identifying and locating nerves by reconstructing a 3D nerve image.

### BACKGROUND OF THE INVENTION

Nerve trend confirmation is necessary for protecting the nerves from damage or being compressed during surgery. For example, when orthopedist implants a locking plate into a patient's limb via an incision to lock broken bone during minimally invasive surgery, the locking plate has to avoid the nerves in implantation because the compression will induce postoperative pain or paralysis.

The nerve locations are varied in different patients, and in order to avoid nerve, orthopedist has to predict the nerve trend during surgery depend on human anatomy and practices only. As a result, nerve compression risk exists in every surgery.

### SUMMARY

One object of the present invention is to confirm nerve location in target region in advance before surgery in order to protect the nerve from damage during surgery (e.g. the nerve is damaged due to the compression of a locking plate).

The method for identifying and locating nerves of the present invention comprising: providing an image display apparatus having a nerve identification program and a nerve identification module; providing a detection apparatus having a detection machine and an image detector mounted on the detection machine; and performing a scanning/identifying procedure. The scanning/identifying procedure includes: a first step of using the image detector to scan a target region to confirm an initial location of a target nerve; a second step of using the image detector to capture a plurality of 2D cross-sectional images of the initial location of the target nerve and using the nerve identification program to identify the 2D cross-sectional images by the nerve identification module and provide a predicted nerve trend to the detection machine; a third step of moving the image detector to a subregion of the target region by the detection machine depend on the predicted nerve trend to capture a plurality of 2D cross-sectional images of the subregion; and a fourth step of using the nerve identification program to determine whether the 2D cross-sectional images of the subregion are captured of the target nerve by the nerve identification module. If the 2D cross-sectional images are identified as images of the target nerve by the nerve identification program, the image detector is moved to the next subregion f the target region by the detection machine according to the predicted nerve trend to scan the target nerve and capture a plurality of 2D cross-sectional images of the next subregion for nerve identification. The fourth step should be performed repeatedly to scan the other subregions when the 2D cross-sectional images are captured of the target nerve.

In the present invention, the 2D cross-sectional images of the target nerve are captured according to the predicted nerve trend by the image display apparatus, the detection apparatus and the scanning/identifying procedure, and a 3D nerve image is created from the 2D cross-sectional images by using 3D image reconstruction technique such that orthopedist can know the target nerve location in advance and the target nerve compressed by locking plate is avoided.

### DESCRIPTION OF THE DRAWINGS

FIG. 1 is a diagram illustrating a radial nerve in an upper limb.

FIG. 2 is a flow chart illustrating a method for identifying and locating nerves in accordance with an embodiment of the present invention.

FIG. 3 is a diagram illustrating an image display apparatus in accordance with an embodiment of the present invention.

FIG. 4 is a perspective assembly diagram illustrating a detection apparatus in accordance with an embodiment of the present invention.

FIG. 5 is a perspective assembly diagram illustrating the detection apparatus in accordance with an embodiment of the present invention.

FIG. 6 is a flow chart illustrating a scanning/identifying procedure in accordance with an embodiment of the present invention.

FIG. 7 is a diagram illustrating 2D images of cross sectional nerve in accordance with an embodiment of the present invention.

FIG. 8 is a diagram illustrating a 3D nerve image in accordance with an embodiment of the present invention.

FIG. 9 is a diagram illustrating a reconstructed image from the 3D nerve image and a target region image in accordance with an embodiment of the present invention.

### DETAILED DESCRIPTION OF THE INVENTION

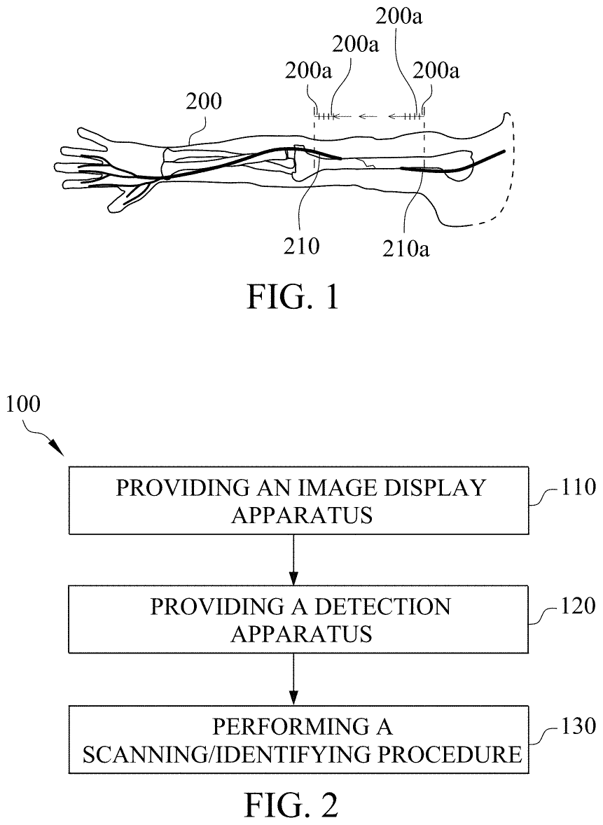

Orthopedist should be careful to avoid compressing radial nerve (cross-sectional area is about 4 mm), median nerve (cross-sectional area is about 4 mm), axillary nerve (cross-sectional area is about 3.6 mm) or ulnar nerve (cross-sectional area is about 3 mm) during minimally invasive surgery. Referring to FIG. 1, as an example, a radial nerve crosses a bone of upper limb, and a locking plate (not shown) is required to fix broken bone, such as radius or humerus of the upper limb bone. However, the radial nerve locations crossing on the bone of upper limb of different patients are varied so as to increase surgical risk.

With reference to FIG. 2, a method for identifying and locating nerves 100 of the present invention is provided to reduce surgical risk. The method 100 includes action 110 of providing an image display apparatus, action 120 of providing a detection apparatus and action 130 of performing a scanning/identifying procedure.

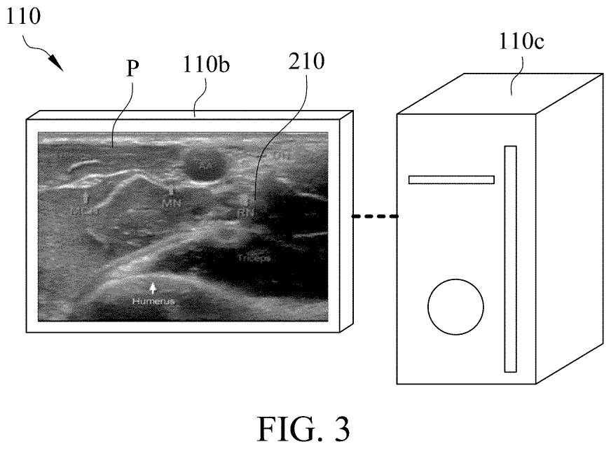

With reference to FIG. 3, an image display apparatus 110a is provided in the action 110.The image display apparatus 110a includes a display 110b and a computer 110c having a nerve identification program and a nerve identification module. The nerve identification module is able to identify the cross section of at least one nerve.

With reference to FIGS. 1, 3, 4 and 5, a detection apparatus 120a is provided in the action 120. The detection apparatus 120a includes a detection machine 120b and an image detector 120c mounted on the detection machine 120b. In this embodiment, the detection machine 120b includes a first track 120d, a second track 120e, a rotation plate 120f, a carrier 120g, a foundation 120h and an accommodation space 120i. The accommodation space 120i is designed to accommodate a target region 200, such as a patient's arm. The first track 120d is arranged on the foundation 120h, the second track 120e is movably connected to the first track 120d and surrounds the accommodation space 120i, and the rotation plate 120f is movably connected to the second track 120e. The carrier 120g having a through hole 120j and a drive rod 120k is coupled to the rotation plate 120f and moved with the rotation plate 120f along the second track 120e. The image detector 120c is mounted on the drive rod 120k inserted in the through hole 120j movably and can be moved toward or away from the target region 200 selectively with the aid of the drive rod 120k. The image detector 120c, may be an ultrasonic detector, is electrically connected to the computer 110c for image signal transmission.

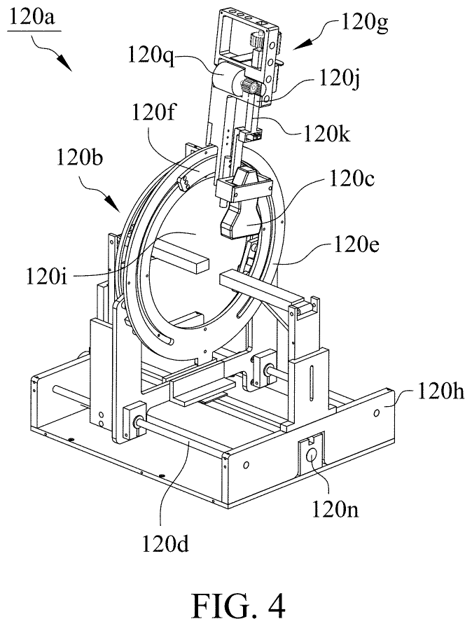

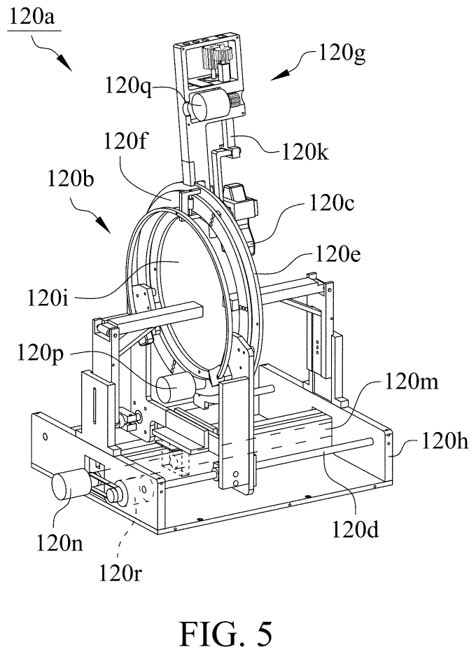

With reference to FIGS. 4 and 5, the detection machine 120b preferably further includes a screw 120m, a first motor 120n, a second motor 120p, a third motor 120q and at least one encoder 120r. The second track 120e is engaged with the screw 120m and the first motor 120n is provided to drive the screw 120m to move the second track 120e along the first track 120d horizontally. The second motor 120p can drive the rotation plate 120f through a pinion and rack assembly or a pulley and belt assembly to move the image detector 120c around the target region 200. The third motor 120q can drive the drive rod 120k with the aid of a pinion and rack assembly or a pulley and belt assembly to selectively move the image detector 120c toward or away from the target region 200.

With reference to FIGS. 4 and 5, the encoder 120r is provided to record drive data of the first motor 120n, the second motor 120p and the third motor 120q, such as the number of revolutions in one direction and the opposite direction. In additional, encoders 120r can be integrated into the first motor 120n, the second motor 120p and the third motor 120q respectively.

With reference to FIGS. 1, 4, 5 and 6, in the action 130, the patient places the target region 200 in the accommodation space 120i firstly, and then the scanning/identifying procedure is performed. At a first step 131, the image detector 120c is used to scan the target region 200 in order to confirm an initial location 210a of the target nerve 210. With reference to FIGS. 1, 3 and 4, a cross-sectional image of the target nerve 210 in the target region 200 is shown on the display 110b by using the image detector 120c to touch and scan the target region 200. In this embodiment, the target nerve 210 is a radius nerve.

An image-capturing frequency parameter is configured to control the number of 2-dimensional (2D) cross-sectional images captured by the image detector 120c during a period of time. For instance, the parameter is configured to capture 300 images in one second. Furthermore, a scanning parameter is configured to control the moving number of the image detector 120c by the detection machine 120b on the target region 200 along a direction. Referring to FIG. 3, a scanning image of the target region 200 captured by the image detector 120c is preferably shown on the display 110b for confirmation.

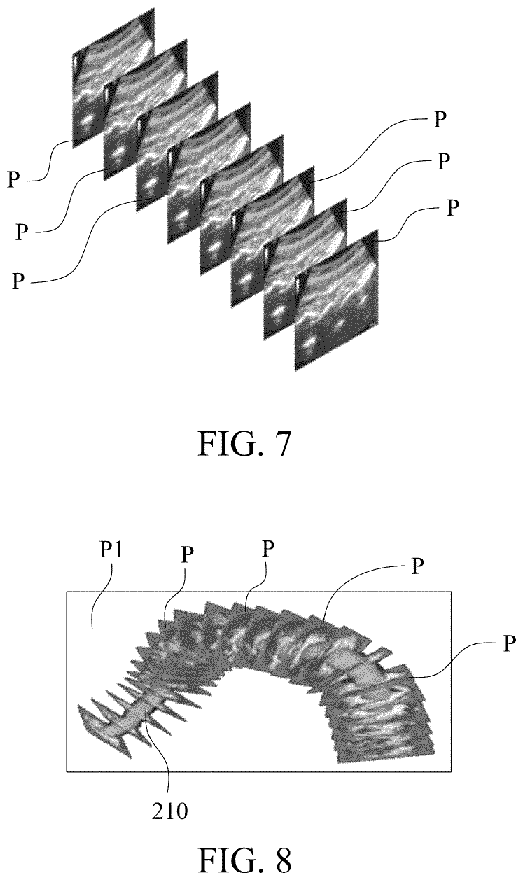

With reference to FIGS. 1, 4, 5 and 6, after the first step 131, a plurality of 2D cross-sectional images P of the initial location 210a of the target nerve 210 (as shown in FIG. 7) are captured by the image detector 120c according to the image-capturing frequency parameter at a second step 132. Each of the 2D cross-sectional images shows a cross section of the target nerve 210. In this embodiment, the image detector 120c can be moved to surround and touch the target region 200 along the second track 120e to capture the 2D cross-sectional images P, and then the 2D cross-sectional images P from the image detector 120c are stored in the computer 110c of the image display apparatus 110a. The nerve identification program identifies the 2D cross-sectional images P by using the nerve identification module and provides a predicted nerve trend to the detection machine 120b. Additionally, during capturing the 2D cross-sectional images P, the drive data of the first motor 120n, the second motor 120p and the third motor 120q are transmitted from the encoder 120r to the computer 110c to be converted into capturing coordinates of the 2D cross-sectional images P by using a operation program, and the capturing coordinates are recorded in the computer 110c. In this embodiment, the capturing coordinate is a displacement coordinate of the image detector 120c moved by the first track 120d, the second track 120e and the drive rod 120k.

With reference to FIGS. 4, 5, 6 and 7, next, according to the predicted nerve trend and the scanning parameter, the detection machine 120b moves the image detector 120c to a subregion 200a within the target region 200 in a third step 133. In this embodiment, the first motor 120n drives the screw 120m in rotation to move the second track 120e, the rotation plate 120f, the drive rod 120k and the image detector 120c to the subregion 200a, then, the image detector 120c is moveed around the target region 200 along the second track 120e to touch the target region 200 and capture a plurality of 2D cross-sectional images P (as shown in FIG. 7) of the subregion 200a based on the image-capturing frequency parameter, and the computer 110c receives the drive data of the first motor 120n, the second motor 120p and the third motor 120q from the encoder 120r and records the capturing coordinates of the 2D cross-sectional images P of the subregion 200a. Identically, the capturing coordinate of each of the 2D cross-sectional images P is a displacement coordinate of the image detector 120c moved to the subregion 200a with the aid of the first track 120d, the second track 120e, the rotation plate 120f and the drive rod 120k.

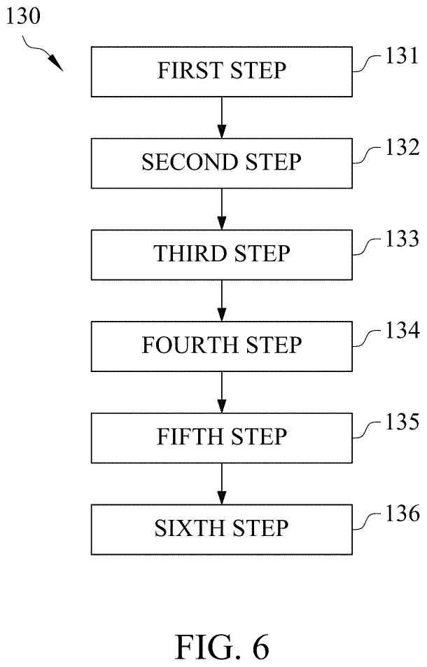

With reference to FIG. 6, at a fourth step 134 next to the third step 133, the nerve identification program determines by the nerve identification module whether the 2D cross-sectional images P, that are captured by the image detector 120c moved to the subregion 200a of the target region 200 in the third step 133, are captured of the target nerve 210. The computer 110c may generate a prompt sign, such as prompt light or prompt sound, while the nerve identification program identifies the 2D cross-sectional images P are the images of the target nerve 210. Later, the image detector 120c is moved by the detection machine 120b to the next subregion 200a of the target region 200 depend on the predicted nerve trend to scan the target nerve 210 and capture multiple 2D cross-sectional images P of the subregion 200a. Also, the computer 110c records the capturing coordinates of the 2D cross-sectional images P and the nerve identification program determines whether the 2D cross-sectional images P of the subregion 200a are captured of the target nerve 210. If the target nerve 210 is shown in the 2D cross-sectional images P, the fourth step 134 is performed repeatedly to scan the other subregions 200a of the target region 200. In contrast, if the nerve identification program determines that the 2D cross-sectional images P captured in the fourth step 134 are not the images of the target nerve 210, it will further provide a new predicted nerve trend to the detection machine 120b to perform the third step 133 and the fourth step 134 again until the image detector 120c completes the scanning operations of the all subregions 200a.

With reference to FIGS. 6, 7 and 8, a fifth step 135 is further involved in the scanning/identifying procedure, in the fifth step 135, a 3-dimensional (3D) image P1 is constructed from the 2D cross-sectional images P captured in the third step 133 and the fourth step 134 based on 3D image reconstruction technique. The 3D image P1 shows a 3D nerve structure, and preferably, the 3D image P1 also shows an alert region surrounding outside the 3D nerve structure.

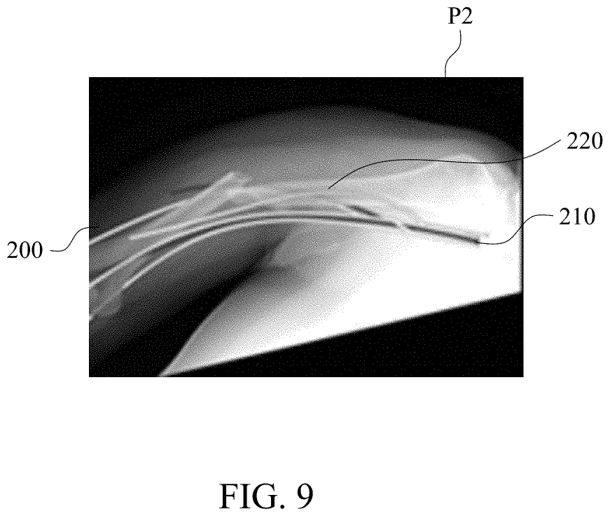

With reference to FIGS. 6, 8 and 9, the scanning/identifying procedure further includes a sixth step 136 next to the fifth step 135. At the sixth step 136, the 3D image P1 is rebuilt onto a target region image P2 captured of the target region 200. As shown in FIG. 9, the target region image P2 is an image of a broken bone 220 in the target region 200, and the rebuilt image is used as reference image to prevent the target nerve 210 from damaging by the locking plate during surgery.

In the present invention, the 2D cross-sectional images P captured of the target nerve 210 are stacked to form the 3D images P1 by using 3D image reconstruction technique such that orthopedist can confirm the location of the target nerve 210 before surgery to sufficiently reduce nerve damage risk.

While this invention has been particularly illustrated and described in detail with respect to the preferred embodiments thereof, it will be clearly understood by those skilled in the art that is not limited to the specific features shown and described and various modified and changed in form and details may be made without departing from the spirit and scope of this invention.

What is claimed is:

1. A method for identifying and locating nerves comprising:
providing an image display apparatus having a nerve identification program and a nerve identification module;
providing a detection apparatus having a detection machine and an image detector mounted on the detection machine; and
performing a scanning/identifying procedure including:
a first step of using the image detector to scan a target region to confirm an initial location of a target nerve;
a second step of using the image detector to capture a plurality of 2D cross-sectional images of the initial location of the target nerve and using the nerve identification program to identify the 2D cross-sectional images by the nerve identification module and provide a predicted nerve trend to the detection machine;
a third step of moving the image detector to one of subregions of the target region by the detection machine depend on the predicted nerve trend to capture a plurality of 2D cross-sectional images of the subregion; and
a fourth step of using the nerve identification program to identify whether the 2D cross-sectional images of the subregion are captured of the target nerve by the nerve identification module,
wherein when the nerve identification program identifies the 2D cross-sectional images are captured of the target nerve, the image detector is moved to the next subregion of the target region by the detection machine depend on the predicted nerve trend to scan the next subregion and capture a plurality of 2D cross-sectional images of the next subregion for identifying whether the 2D cross-sectional images of the next subregion are captured of the target nerve,
and wherein when the 2D cross-sectional images of the next subregion are captured of the target nerve, the fourth step is performed repeatedly to scan the other subregions by using the image detector.

2. The method in accordance with claim 1, wherein a cross-sectional nerve image of the target region is shown on a display when the image detector is moved to touch the target region.

3. The method in accordance with claim 1, wherein when the nerve identification program identifies the 2D cross-sectional images are not captured of the target nerve at the fourth step, the nerve identification program provides a new predicted nerve trend to the detection machine to perform the third and fourth steps for scanning the other subregions by using the image detector.

4. The method in accordance with claim 1, wherein a prompt sign is generated when the nerve identification program identifies the 2D cross-sectional images are captured of the target nerve, the prompt sign is a prompt light or a prompt sound.

5. The method in accordance with claim 1, wherein the scanning/identifying procedure further includes a fifth step of constructing a 3D image showing a 3D nerve structure from the 2D cross-sectional images captured at the third and fourth steps by using a 3D image reconstruction technique.

6. The method in accordance with claim 5, wherein the 3D image shows an alert region surrounding outside the 3D nerve structure.

7. The method in accordance with claim 1, wherein the scanning/identifying procedure further includes a sixth step of rebuilding the 3D image constructed at the fifth step onto a target region image of the target region.

8. The method in accordance with claim 1, wherein each of the 2D cross-sectional images shows a cross section of the target nerve.

9. The method in accordance with claim 1, wherein a capturing coordinate of each of the 2D cross-sectional images is recorded during capturing each of the 2D cross-sectional images.

10. The method in accordance with claim 9, wherein the detection machine includes a first track, a second track, a rotation plate and a carrier, the second track is moveably connected to the first track, the rotation plate is movably connected to the second track, the carrier having a through hole and a drive rod is connected to the rotation plate and swing with the rotation plate, the drive rid is movably inserted in the through hole, and the image detector is mounted on the drive rod, wherein the capturing coordinate is a displacement coordinate of the image detector moved by the second track, the rotation plate and the drive rod.

11. The method in accordance with claim 10, wherein the detection machine further includes a foundation and an accommodation space for accommodating the target region, the first track is mounted on the foundation, the second track surrounds the accommodation space and the image detector is able to be moved toward or away from the target region by the drive rod.

12. The method in accordance with claim 11, wherein the detection machine further includes a screw and a first motor, the second track is engaged with the screw, and the first motor is used to drive the screw in rotation to move the second track horizontally along the first track.

13. The method in accordance with claim 12, wherein the detection machine further includes a second motor used to drive the rotation plate to swing such that the image detector is able to he moved around the target region.

14. The method in accordance with claim 13, wherein the detection machine further includes a third motor used to drive the drive rod to move the image detector toward or away from the target region selectively.

15. The method in accordance with claim 14, wherein the detection machine further includes at least one encoder used to record drive data of the first, second and third motors, and the drive data is converted into the capturing coordinate by an operation program.

16. The method in accordance with claim 1, wherein an image-capturing frequency parameter and a plurality of scanning parameters are configured in the image detector at the first step.

17. The method in accordance with claim 16, wherein the image detector is used to capture the 2D cross-sectional images of the initial location of the target nerve depend on the image-capturing frequency parameter at the second step.

18. The method in accordance with claim 16, wherein the image detector is moved to the subregion of the target region by the detection machine depend on the predicted nerve trend and the scanning parameters and is used to capture the 2D cross-sectional images of the subregion depend on the image-capturing frequency parameter at the third step.
